(19) United States
(12) Plant Patent Application Publication
(10) Pub. No.: US 2017/0112035 P1
(43) Pub. Date: Apr. 20, 2017

(54) LACHENALIA JACQ. F. EX MURRAY named "CHERISE"

(50) ;

(71) Applicant: AGRICULTURAL RESEARCH COUNCIL, Pretoria (ZA)

(72) Inventor: Riana Kleynhans, Benoni (ZA)

(21) Appl. No.: 14/756,801

(22) Filed: Oct. 15, 2015

(51) Int. Cl. A01H 5/00

(52) U.S. Cl. PLT/263.1

(57) ABSTRACT

A new and distinct form of Lachenalia plant characterized by a bulb plant type having a spreading growth habit of a height of 15-30 centimeters (cm) and width of 10-15 cm having a cerise pink flower and green leave with maroon spots.

Genus and species: Lachenalia Jacq. f. ex Murray.

Variety denomination: Cherise.

### BACKGROUND OF THE INVENTION

The present invention relates to a new and distinct cultivar of Lachenalia, botanically known as Lachenalia Jacq. f. ex Murray having the common name of Viooltjies/Cape cowslip and hereinafter referred to by its variety denomination “Cherise”.

The variety was first cultivated in 2008 and originated by means of conventional breeding by the inventor during 2009 to 2010 at the premises of Agricultural Research Council Vegetable and Ornamental Plant Institute, Roodeplaat, Pretoria, Republic of South Africa.

Asexual reproduction of the Cherise plant has shown that the unique features thereof are stable and reproduced true to type in successive generations.

### SUMMARY OF THE INVENTION

The present invention constitutes a new and distinct variety of Lachenalia which originated from the controlled pollination between a Lachenalia hybrid as seed bearing parent and a Lachenalia hybrid as pollen parent as detailed hereinbelow.

The following traits have been repeatedly observed and are determined to be the unique characteristics, under the local evaluation conditions, of the Cherise plant. These characteristics in combination distinguish Cherise as a new and distinct Lachenalia cultivar:
1. Bulb plant type having a spreading growth habit of a height of 15-30 centimeters (cm) and width of 10-15cm.
2. Flower color: cerise pink.
3. Leaf color: green with maroon spots.

Plants of the new Lachenalia variety Cherise can be compared to similar denominations such as Robyn and Rosabeth but differs in that the flower color of Cherise is cerise pink compared to the red and red with green tips of Robyn and Rosabeth respectively.

### BRIEF DESCRIPTION OF THE PHOTOGRAPHS

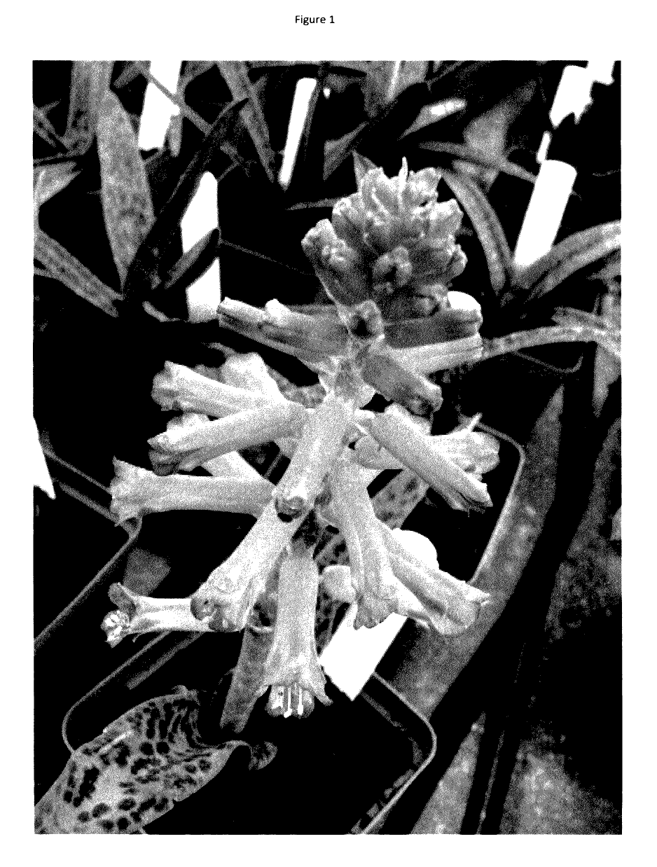

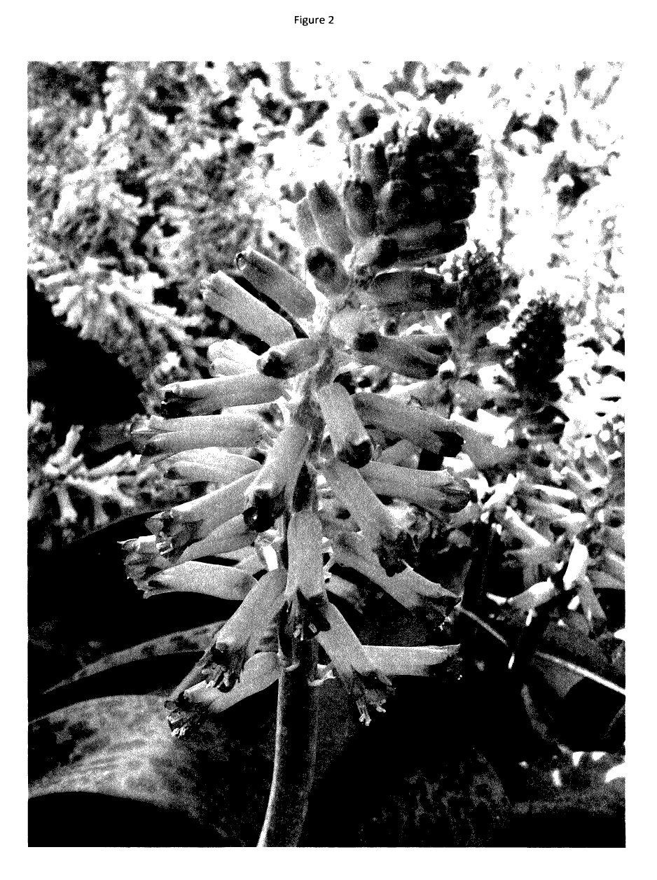

The accompanying colored photographs illustrate the overall appearance of the Cherise, showing the colors as true as it is reasonably possible to obtain in colored reproductions of this type. The photographs comprise front perspective views of a typical Cherise grown in an indoor nursery (FIG. 1), and one of which is located in an outdoor setting (FIG. 2).

### DETAILED DESCRIPTION

The aforementioned photographs, following observations and measurements describe Cherise grown by the inventor on behalf of Agricultural Research Council Vegetable and Ornamental Plant Institute, Roodeplaat, Pretoria, Republic of South Africa. Thereafter Cherise was made subject of a South African Plant Breeders' Right application in March 2009. This application was granted in 2010 under number ZA20104393.

The plant is a late flowering cultivar and may be used for landscaping and should be planted during the month of March; indoors and in pots only. The plant should be kept at temperatures of 20-25 degrees Celsius during the day and 10-15 degrees Celsius at night. Plenty of light should be provided.

Cherise is a hybrid-hybrid cross containing the following Lachenalia species: Lachenalia orchioides subsp. glaucina, Lachenalia bulbifera, Lachenalia quadricolor, Lachenalia rubida, Lachenalia abides.

The specific Cherise parentage is as follows:
Female parent: (L. bulbifera×[L. aloides×L. rubida])×([L. bulbifera×L. rubida]×L. bulbifera).
Male parent: {([L. orchioides×L. bulbifera]×[L. orchioides×L. bulbifera])×([L. orichioides×L. rubida]×[L. orichioides×L. rubida])}×{([L. aloides×L. rubida]×[L. aloides×L. rubida])×([L. orchioides×L. bulbifera]×[L. orchioides×L. bulbifera])}.
Leaf: Monocolored. Spots on upper side — Present.
Flower:
   Predominant color.—Cerise.
   Intensity of predominant color.—Medium to strong.
   Outer perianth segment.—Color of apex relative to other part. Clearly different.
   Inner perianth segment.—Color of margin of apex relative to other part. Clearly different.
   Attitude.—Horizontal.
   Flower length.—3.2 cm.
   Pedicel.—Present: length medium.
   Flower diameter excluding apex.—Small to medium: 0.6 cm.
   Flower diameter excluding apex.—Medium 0.8 cm.
   Flower attitude of distal part of inner perianth segments.—Spreading.
   Flower glossiness.—Absent.
   Outer perianth segment.—Color of basal part just before opening of flower: Red (RHS # 47D).
   Outer perianth segment.—Color of basal part of fully opened flower: Red (RHS # 47C).
   Outer perianth segment.—Color of apex relative to other part: Clearly different (yellow-brown).
   Inner perianth segment.—Distinct spot in middle of apex: Present.
   Inner perianth segment.—Color of margin of apex relative to other part: Clearly different (purple).
   Inner perianth segment.—Predominant color of exposed part (excluding differently colored margin of apex). Red (RHS # 47C).
   Length of inner perianth segment compared to outer segments.—Longer. The variety is not genetically modified and has been tested to be free of Ornithogalum Mosaic Virus.

The invention claimed is:

1. A new and distinct cultivar of Lachenalia named Cherise as illustrated and described herein.
